(19) United States
(12) Patent Application Publication
(10) Pub. No.: US 2025/0134772 A1
(43) Pub. Date: May 1, 2025

(54) CLOSED-LIQUID TRANSFER DEVICES AND SYSTEMS

(71) Applicant: West Pharma. Services IL, Ltd., Ra'anana (IL)

(72) Inventors: Elinor ZAROUR SHALEV, Ramat Hasharon (IL); Elisheva FABRIKANT, Herzliya (IL); Edan SHALEV, Kfar Saba (IL); Niv BEN SHALOM, Netanya (IL); Gil YIGAL, Gan Yavne (IL); Yossi BAR EL, Beit Arieh (IL); Amir LEV, Kfar Saba (IL); Moshe GILBOA, Kfar Saba (IL); Igor ISAKOV, Rehovot (IL)

(21) Appl. No.: 18/836,455

(22) PCT Filed: Feb. 18, 2023
(86) PCT No.: PCT/IB2023/051481

§ 371 Date: Aug. 7, 2024

Related U.S. Application Data
(60) Provisional application No. 63/311,802, filed on Feb. 18, 2022.

(51) Int. Cl. A61J 1/20

(52) U.S. Cl. CPC A61J 1/2058; A61J 1/201

(57) ABSTRACT

A closed-system liquid transfer device is used with each of an infusion liquid container containing an infusion liquid, a medicament supply containing a medicament, and an infusion set. The closed-system liquid transfer device includes first, second, and third connectors that respectively connect and resist removal from the infusion liquid container, the infusion set, and the medicament supply.

### CROSS REFERENCE TO RELATED APPLICATIONS

This application claims the benefit of U.S. Provisional Patent App. No. 63/311,802, filed Feb. 18, 2022, the disclosure of which is hereby incorporated by reference herein.

### TECHNICAL FIELD

The present invention generally relates to closed-liquid transfer devices and systems, particularly for secure transfer of a medicament to an infusion container and from the infusion container to an infusion set.

### BACKGROUND

Closed sterile transfer devices (CSTDs) can be important for use with medicaments (e.g., drugs, vaccines, etc.), including for certain chemotherapy drugs. CSTDs can limit the possibility of exposure to the person preparing or administering hazardous medicaments. Devices exist allowing various medicaments to be transferred to an infusion container and from the infusion container to an infusion set for intravenously supplying the medicament together with infusion fluid to a patient. For example, medicaments can be supplied to an infusion container via a syringe that connects to a fitting. The fitting can be connected to the infusion container, the medicament supply, and to the infusion set. However, the connections between the fitting and each of the infusion container, the medicament supply, and the infusion set can be insufficiently strong and can be removed when subject to relatively small removal forces. This can present security or contamination concerns particularly when the medicament is hazardous.

Accordingly, there exists a need for a closed-liquid transfer device that securely and universally connects to infusion containers, medicament supplies, and infusion sets and that can resist removal of these connections to prevent tampering with the medicament and/or infusion liquid. There also exists a need for one or more of connections that allow for closed sterile transfer of medicaments.

### SUMMARY

These needs are met, to a great extent, by a closed-system liquid transfer device configured for use with each of an infusion liquid container containing an infusion liquid and having a port for administering the infusion liquid. The closed-system liquid transfer device includes a body. The device includes an IV set spike at a first end of the body configured to be fluidly connected to the port of the infusion liquid container. The device also includes a port at a second end of the body that may include a locking ring configured to sealingly receive the IV set spike of the infusion set and to resist a removal force applied to the port of the closed-system liquid transfer device or to the IV set spike of the infusion set. The device also includes and a female Luer at a third end of the body configured to be fluidly connected to the male Luer of the medicament supply.

Implementations may include one or more of the following features. The locking ring can have a plurality of circumferential ridges. The plurality of circumferential ridges may be provided on an inner surface of the locking ring and the locking ring may be provided within a recess of the port of the closed-system liquid transfer device. The recess may be tapered. Translation of the locking ring towards a narrow portion of the recess may reduce a diameter of the locking ring. The locking ring may include a lateral slit. The female Luer may include a plurality of protrusions that are configured to resist rotation of the male Luer in an opening direction. The plurality of protrusions may be arranged in a circumferential thread. The IV set spike of the closed-system liquid transfer device may include a restrainer having an outwardly projecting trailing edge. The outwardly projecting trailing edge may define a plurality of barbs. The IV set spike of the closed-system liquid transfer device may include: a tip; and one or more ribs between the tip, and the restrainer. The IV set spike of the closed-system liquid transfer device may include an abutment, and an opposing end of the restrainer relative to the outwardly projecting trailing edge may be adjacent to the abutment. The second lumen may be configured to provide fluid communication from the infusion liquid container to the port of the closed-system liquid transfer device including liquid received from the medicament supply via the first lumen. The locking ring may have a topside and a bottom side. The topside has a top projection that is substantially perpendicular to the plane of topside and the bottom side has a bottom projection that is substantially perpendicular to the plane of the bottom side and in a 180° direction opposite the top projection and on opposite portions of the locking ring. The bottom projection and the top projection can be of different lengths. The locking ring can be planar and can be positioned within a holder having an opening at its bottom sized to accommodate insertion of the IV set spike of the infusion set. The holder can have an inner wall and an interior step. The inner wall can have a larger diameter than a diameter of the opening and the interior step can between the opening and the inner wall. The locking ring can be on top of the interior step and a spring is on top of the locking ring. The interior step does not extend more than 25% of the circumference of the inner wall and the locking ring has an outer circumference that is no more than 10% smaller than the inner wall circumference. The holder is a first holder and the closed-system liquid transfer device further comprises a second holder received within the first holder. The first holder comprises a first connector and the second holder comprises a second connector that is complementary to the first connector and that is configured to connect with the first connector to connect the first holder and the second holder. The first holder comprises a first stopper and the second holder comprises a second stopper that is complementary to the first stopper and that is configured to engage with the first stopper to prevent relative rotation between the first holder and the second holder. The IV set spike of the closed-system liquid transfer device comprises arms defining teeth that are configured to grip the port of the infusion liquid container. A kit may include the closed-system liquid transfer device, the infusion liquid container, the medicament supply, and the infusion set. The medicament supply may include a syringe.

One general aspect includes a closed-system liquid transfer device configured for use with each of an infusion liquid container containing an infusion liquid and having a port for administering the infusion liquid. The closed-system liquid transfer device includes a body. The device also includes an IV set spike at a first end of the body configured to be fluidly connected to the port of the infusion liquid container. The device also includes a port at a second end configured to sealingly receive the IV set spike of the infusion set. The device also includes and a female Luer at a third end of the body configured to be fluidly connected to the male Luer of the medicament supply, the female Luer may include a plurality of a plurality of protrusions that are configured to resist rotation of the male Luer in an opening direction.

Implementations may include one or more of the following features. The plurality of protrusions may be arranged in a circumferential thread. The female Luer further comprises a guard. The IV set spike of the closed-system liquid transfer device may include a restrainer having an outwardly projecting trailing edge. The outwardly projecting trailing edge may include a plurality of barbs. The IV set spike of the closed-system liquid transfer device may include: a tip; and one or more ribs between the tip and the restrainer. The IV set spike of the closed-system liquid transfer device may include an abutment and an opposing end of the restrainer relative to the outwardly projecting trailing edge may be adjacent to the abutment. The second lumen may be configured to provide fluid communication from the infusion liquid container to the port of the closed-system liquid transfer device including liquid received from the medicament supply via the first lumen. A kit may include the closed-system liquid transfer device, the infusion liquid container, the medicament supply, and the infusion set. The medicament supply may include a syringe.

One general aspect includes a fitting with a first connector that is configured to fluidly connect the fitting to a port of an IV bag, the first connector may include a first opposer that is configured to resist a first force applied to the first connector or to the port, where the first force is at least 30 N. The fitting also includes a second connector that is configured to fluidly connect the fitting to an IV set spike of an infusion set, the second connector may include a second opposer that is configured to resist a second force applied to the second connector or to the IV set spike, where the second force is at least 30 N. The fitting also includes a third connector that is configured to fluidly connect the fitting to a male Luer of a medicament supply, the second connector may include a third opposer that is configured to resist a third force applied to the second connector or to the male Luer, where the third force is at least 30 N. The fitting also includes a body that fluidly connects and of the first, second, and third connectors.

Implementations may include one or more of the following features. The first connector may define an IV set spike and the first opposer may define a restrainer having an outwardly projecting trailing edge that is configured to embed into the port of the IV bag upon application of the first force. The second connector may define a port and the second opposer may define a locking ring that is configured to grip the IV set spike of the infusion set. The third connector may define a female Luer and the third opposer defines protrusions that are configured to resist rotation of the male Luer in an opening direction.

One general aspect includes a device for closed sterile transfer of a substance. The device includes an outer body defining an interior and a channel extending through the outer body and opening into the interior. The device includes a septum that seals the channel of the outer body, and an inner body within the interior of the outer body. The inner body defining an interior. The inner body may include: a seal within the interior of the inner body that is configured to fluidly seal an opening of a container; a projection extending through the seal; and a filter. The projection may include: a first channel that fluidly connects the channel of the outer body and the interior of the inner body; and a second channel that fluidly connects the interior of the outer body and the interior of the inner body. The inner body and the outer body together define a pathway for gas to exit from the interior of the inner body, the pathway extends through the second channel of the inner body, through the interior of the outer body, through the filter, and out of an outlet of the device.

Implementations may include one or more of the following features. The device may include the container, where the opening of the container may include at least one of a cyclic olefin polymer or a cyclic olefin copolymer. The filter extends circumferentially about a central axis of the inner body. The filter may include hydrophobic media that prevents fluid from exiting through the outlet. The filter is charged. The filter may include pores having a size of 0.2 μm or less. The filter may include PTFE. The filter is pleated. The filter is resilient and is configured to exert a force on the seal to fluidly seal the container opening. The device may include a cap that is configured to be removably attached to the outer body to cover the septum.

Various additional features and advantages of this invention will become apparent to those of ordinary skill in the art upon review of the following detailed description of the illustrative embodiments taken in conjunction with the accompanying drawings.

### BRIEF DESCRIPTION OF THE DRAWINGS

The following detailed description is better understood when read in conjunction with the appended drawings. For the purposes of illustration, examples are shown in the drawings; however, the subject matter is not limited to the specific elements and instrumentalities disclosed. In the drawings:

FIG. 1 shows a schematic representation of a closed-liquid transfer system;

FIG. 2 shows a first embodiment of a first connector;

FIG. 3 shows a cross section view of the first embodiment of the first connector fit to an IV bag;

FIG. 4 shows a second embodiment of the first connector;

FIG. 5 shows a third embodiment of the first connector;

FIG. 6 shows a fourth embodiment of the first connector;

FIG. 7 shows a fifth embodiment of the first connector;

FIG. 8 shows the fifth embodiment of the first connector connected to a rigid port;

FIG. 9 shows the fifth embodiment of the first connector connected to a flexible port;

FIG. 10 shows a first embodiment of a second connector;

FIG. 11 shows a locking ring of the first embodiment of the second connector;

FIG. 12 shows an exploded view of a second embodiment of the second connector;

FIG. 13 shows a cross section view of the second embodiment of the second connector;

FIG. 14 shows a cross section view of a third embodiment of the second connector;

FIG. 15 shows a cross section view of a third embodiment of the second connector upon insertion of an IV set spike;

FIG. 16 shows a cross section view of a third embodiment of the second connector with a locking ring engaged with the IV set spike;

FIG. 17 shows a cross section view of the fourth embodiment of the second connector;

FIG. 18 shows a view of fifth embodiment of the second connector;

FIG. 19 shows a cross section view of a first embodiment of the third connector;

FIG. 20 shows a cross section view of a second embodiment of the third connector;

FIG. 21 shows a first embodiment of the fitting;

FIG. 22 shows a second embodiment of the fitting;

FIG. 23 shows an exploded view of a device for closed sterile transfer; and

FIG. 24 shows a gas traveling through a pathway of the device for closed sterile transfer of FIG. 23.

### DETAILED DESCRIPTION

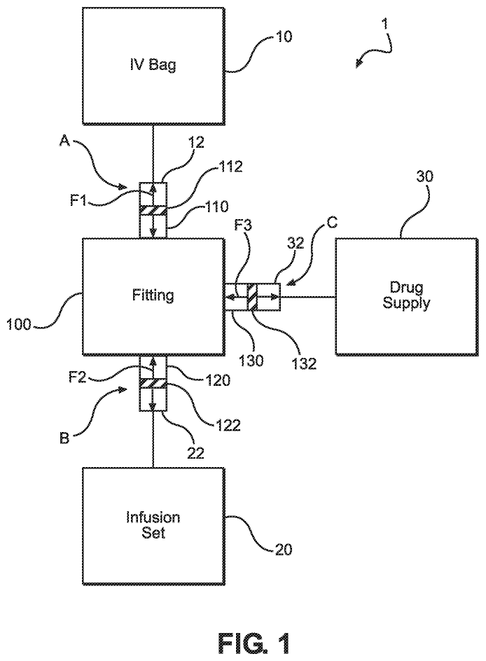

FIG. 1 illustrates a closed-liquid transfer system 1 for intravenously delivering a medicament. System 1 can be a kit. System 1 can include an infusion liquid container, e.g., an intravenous (IV) bag 10, that can contain an infusion liquid such as saline or any other fluid that can be administered intravenously to a patient. System 1 can include an infusion set 20 that can supply infusion fluid from IV bag 10 and/or a medicament to a patient for intravenously administering the infusion fluid and/or medicament. System 1 can include a medicament supply 30 (e.g., a container with a syringe, a vial, etc.) that can contain a medicament that can be administered intravenously to a patient alone and/or in combination with infusion fluid. The term “medicament” as used herein includes the plain and ordinary meaning and can also encompass any other substance that can be administered intravenously to a patient alone or in combination with infusion fluid including for example vitamins, nutrients, electrolytes, among others.

System I also includes a fitting 100 having a body that can connect fluidly connect (i.e., physically connect in a manner that permits fluid to flow from one structure to the other) to any and all of IV bag 10, infusion set 20, and medicament supply 30. Fitting 100 can be a closed-system liquid transfer device. Fitting 100 can include a monolithic trifurcated connector body defining a barrel and that can be fluidly connected to each of a first connector 110, a second connector 120, and a third connector 130, described further below. Fitting 100 can be formed of materials compatible with medicaments and infusion fluid can flow through fitting 100. Fitting 100 can provide a closed-liquid transfer device that securely and universally connects to infusion containers, medicament supplies, and infusion sets and that can resist removal of these connections to prevent tampering with the medicament and/or infusion liquid. Further, since the features that secure the connections are a part of fitting 100, connections to existing infusion containers, medicament supplies, and infusion sets can be secured without modification to the existing infusion containers, medicament supplies, and infusion sets. This can also improve the comfort level of medical personnel administering medicaments since the connections will be familiar but secured.

As shown in FIG. 1, fitting 100 and IV bag 10 can be fluidly connected at connection A to permit fluid to flow from IV bag 10 to body of fitting 100. For example, IV bag 10 can include a port 12, which can be sized to comply with any IV port standards such as those set by the international organization for standardization (ISO). Fitting 100 can include a first connector 110 that can connect to port 12 to form connection A. The first connector 110 can include a first opposer 112 that can resist a force F1 that can be applied directly or indirectly to first connector 110 and/or port 12 in one or more directions to pull connection A apart, as shown in FIG. 1. That is, first opposer 112 can resist the force F1 to keep connection A together. First opposer 112 can resist force F1 up to at least 15 N.

In embodiments, first connector 110 can be an IV set spike. Fitting 100 can include a first lumen that can fluidly connect an opening of third connector 130 with a first distally disposed opening of the first connector 110. Fitting 100 can include a second lumen that can fluidly connect second connector 120 with a second distally disposed opening of the first connector 110. According to this configuration, communication between the third connector 130 and the IV set spike can be separated from fluid communication between the second connector 120 and the IV set spike. This can enable introduction of a medicament from the medicament supply 30 to the IV bag 10 through the third connector 130 and the first lumen for mixing with the infusion liquid to form a medicated infusion liquid. This can also enable subsequent administration of the medicament-infused liquid to a patient from the IV bag through the second lumen and the second connector 120 to the infusion set 20.

First opposer 112 can include one or more ribs that protrude from the first connector 110. Additionally or alternatively, opposer can include a restrainer that can be snap fit around first connector 110. Restrainer can include one or more outwardly directed tabs or barbs. For example, restrainer can include a circumferential array of outwardly directed barbs.

Returning to FIG. 1, fitting 100 and infusion set 20 can be fluidly connected at connection B to permit fluid to flow from the body of fitting 100 to the infusion set 20. For example, infusion set 20 can include an IV set spike 22, which can be sized to comply with any IV set spike standards such as those set by ISO. For example, IV set spike 22 can have an external diameter of 5 mm. Fitting 100 can include a second connector 120 that can connect to IV set spike 22 to form connection B. Second connector 120 can include a second opposer 122 that can resist a force F2 that can be applied directly or indirectly to second connector 120 and/or IV set spike 22 in one or more directions to pull connection B apart, as shown in FIG. 1. That is, second opposer 122 can resist force F2 to keep connection B together. Second opposer 122 can resist force F2 up to at least 15 N.

In embodiments, second connector 120 can be a port that receives IV set spike 22. Second opposer 122 can be a locking ring disposed within the port. Locking ring can include inner circumferential ridges that can grip the IV set spike 22. Locking ring can be made of any suitable material including for example plastic and/or metal. Second connector 120 and/or second opposer 122 can include a taper. For example, second connector 120 and/or second opposer 122 can taper from an opening of second connector 120 and/or second opposer 122.

Returning again to FIG. 1, fitting 100 and medicament supply 30 can be fluidly connected at connection C to permit fluid to flow from medicament supply 30 to body of fitting 100. For example, medicament supply 30 can include a male Luer 32, which can be sized to comply with any male Luer standards such as those set by ISO. Fitting 100 can include a third connector 130 that can connect to male Luer 32 to form connection C. Third connector 130 can include a third opposer 132 that can resist a force F3 that can be applied directly or indirectly to third connector 130 and/or male Luer 32 in one or more directions to pull connection C apart, as shown in FIG. 1. That is, third opposer 132 can resist force F3 to keep connection C together. Third opposer 132 can resist force F3 up to at least the minimum standards as required by ISO 11040-4 Annex C2.

In embodiments, third connector 130 can be a female Luer that receives male Luer 32. Female Luer can include threads. Third opposer 132 can be a ring. Ring can include protrusions such as fins. Protrusions can be flexible in one direction (e.g., a closing direction) and rigid in another direction (e.g., an opening direction). According to this configuration a locking nut of male Luer 32 can be threaded onto third connector 130 and third opposer 132 can resist removal of the locking nut of male Luer 32.

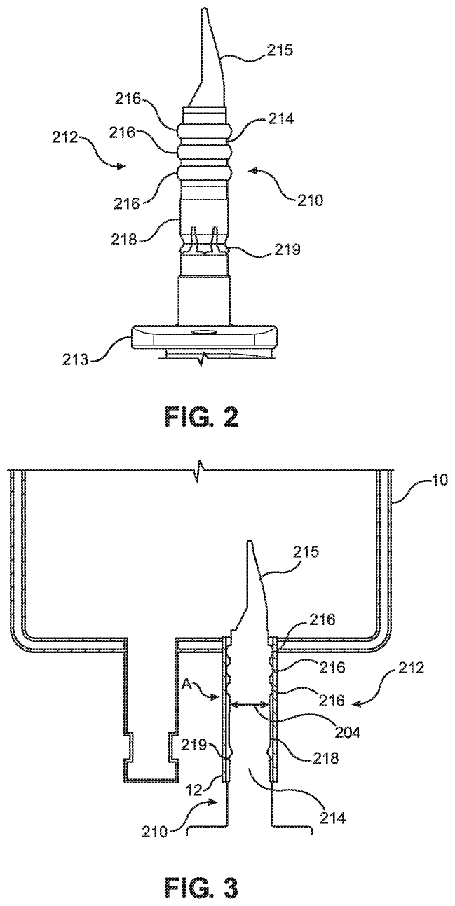

FIGS. 2-9 show embodiments of first connectors 210, 310, 410, 510, and 610. Except for mutually exclusive features, embodiments of the first connectors 210, 310, 410, 510, and 610 can include any of the features previously described in reference to the first connector 110, where like parts correspond to like parts, and vice versa. Further, any of the first connectors 210, 310, 410, 510, and 610 can be used in the system 1 and can be provided as a part of the fitting 100 in the same manner described previously in reference to the first connector 110.

FIGS. 2 and 3 show views of the first connector 210. The first connector 210 can include a flange 213 and an IV set spike 214 extending proximally from the flange 213. As shown in FIG. 3, the IV set spike 214 can be inserted through the port 12 of the IV bag 10. The IV set spike 214 can include an external diameter 204. The IV set spike 214 can taper from external diameter 204 to a tip 215 of the IV set spike 214. The tip 215 can puncture a septum of the port 12 upon sliding insertion into the port 12 to open the IV bag 10. The external diameter 204 of the IV set spike 214 can be less than 5.6 mm. For example, the external diameter 204 of IV set spike 214 can be 4.5 mm (+/−10%).

The first connector 210 can include an opposer 212 provided on the IV set spike 214. The opposer 212 can include one or more ribs 216. The ribs 216 can protrude from the IV set spike 214. The ribs 216 can form a seal to prevent fluid from escaping the IV bag 10 between the IV set spike 214 and the port 12. The ribs 216 can be formed integrally with the IV set spike 214. Alternatively, the ribs 216 can be formed separately from the IV set spike 214 and subsequently fixed to IV set spike 214 for example with adhesive or other known fixing techniques. The ribs 216 can be formed of a material that can contact infusion fluid and/or a medicament without contaminating the infusion fluid or the medicament. The ribs 216 can be formed of the same material that forms the IV set spike 214. Alternatively, the ribs 216 can be formed from different materials than the IV set spike 214. The ribs 216 can continuously surround the IV set spike 214. Alternatively, the ribs 216 can discontinuously surround the IV set spike 214. In embodiments, the first connector 210 can include three ribs 216, though any number of ribs 216, including one rib 216 or no ribs, are possible.

In embodiments, the opposer 212 can additionally or alternatively include a restrainer 218. The restrainer 218 can discontinuously surround the IV set spike 214. For example, the restrainer 218 can include a longitudinal slit that can allow the restrainer 218 to be snap fit around the IV set spike 214. Alternatively, the restrainer 218 can continuously surround the IV set spike 214.

The restrainers 218 can include an outwardly projecting trailing edge 219. The trailing edge 219 can taper in a direction towards the tip 215 of the IV set spike 214 and can flare out angularly beyond an external surface of the IV set spike 214 away from the tip 215. The trailing edge 219 can be elastically deflectable. For example, the trailing edge 219 can elastically deflect against the IV set spike 214 when the IV set spike 214 is inserted into the port 12 and the trailing edge 219 can elastically deflect radially away from the IV set spike 214 when the IV set spike 214 is pulled outwardly relative to the port 12. According to this arrangement, the trailing edge 219 can grip and/or embed into an interior of the port 12 to resist removal of the IV set spike 214 without significantly interfering with insertion of the IV set spike 214 into the port 12. The trailing edge 219 can include one or more tabs. Additionally or alternatively, the trailing edge 219 can include one or more barbs.

The restrainer 218 can be formed of a different material than a material that forms the IV set spike 214. For example, the IV set spike 214 can be formed of a material that can contact infusion fluid and/or a medicament without contaminating the infusion fluid or the medicament. The restrainer 218 can be formed of a different material, such as stainless steel, since restrainer 218 can be arranged on the IV set spike 214 without contacting infusion fluid and/or a medicament. For example, the restrainer 218 can be arranged on the IV set spike 214 with the ribs 216 interposed between the tip 215 and the restrainer 218 such that the ribs 216 prevent infusion fluid and/or medicament from contacting be restrainer 218. Alternatively, the restrainer 218 can be formed of the same material that forms the IV set spike 214.

In embodiments, the restrainer 218 can indicate sufficient insertion of the IV set spike 214 into the port 12. For example, the port 12 can be transparent and a user can monitor a distance between the restrainer 218 and a rim of the port 12 to ensure the IV set spike 214 has been sufficiently inserted into the port 12. The distance between the restrainer 218 and a rim of port 12 can for example be at least 2 mm.

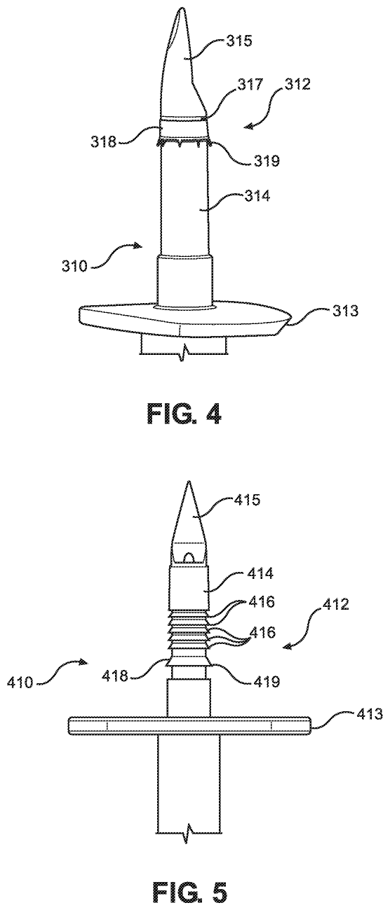

FIG. 4 shows a perspective view of the first connector 310. Except for mutually exclusive features, the first connector 310 can include any or all of the features described previously in reference to the first connector 210 where like parts correspond to like parts, and vice versa. For example, the first connector 310 can include an opposer 312, a flange 313, an IV set spike 314, a tip 315, a restrainer 318 having a trailing edge 319. The restrainer 318 can be arranged adjacent to the tip 315. For example, the restrainer 318 can be arranged distally relative to the midpoint of the IV set spike 314. The trailing edge 319 can include a plurality of barbs. The IV set spike 314 can include an abutment flange 317. The abutment flange 317 can be disposed between tip 315 and restrainer 318. The abutment flange 317 can limit movement of the restrainer 318 towards the tip 315 to prevent restrainer 318 from sliding off the IV set spike 314 when a removal force is applied to IV set spike 314, such as force FI shown in FIG. 1 and described previously. In embodiments, the first connector 310 can be provided without ribs. Alternatively, the first connector 310 can include any number of ribs.

FIG. 5 shows a perspective the first connector 410. Except for mutually exclusive features, the first connector 410 can include any or all of the features described previously in reference to the first connectors 210, 310 where like parts correspond to like parts, and vice versa. For example, the first connector 410 can include an opposer 412, a flange 413, an IV set spike 414, a tip 415, one or more ribs 416, and a restrainer 418 having a trailing edge 419. In embodiments, the ribs 416 and/or the restrainer 418 can be formed integrally with the IV set spike 414. In embodiments, the first connector 410 can include five ribs 416, though other numbers of ribs or no ribs are possible. The restrainer 418 and one or more of the ribs 416 can be provided proximally relative to the midpoint of the IV set spike 414.

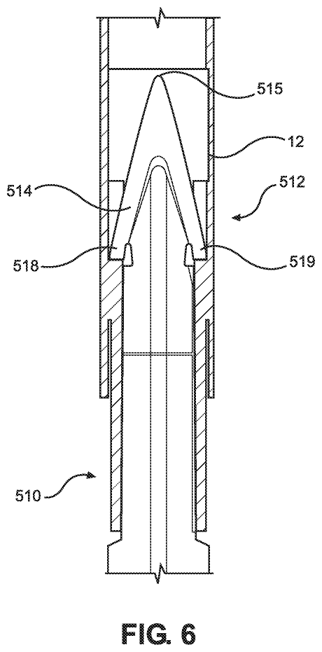

FIG. 6 shows a cross-section view of the first connector 510 disposed within the port 12. Except for mutually exclusive features, the first connector 510 can include any or all of the features described previously in reference to the first connectors 210, 310, 410 where like parts correspond to like parts, and vice versa. For example, the first connector 510 can include an opposer 512, a flange (not shown), an IV set spike 514, a tip 515, and a restrainer 518 having a trailing edge 519. The restrainer 518 can be harpoon-shaped. The trailing edge 519 can hook into an inner membrane of the port 12 to secure the IV set spike 514 to the port 12. In embodiments, the first connector 510 can be provided without ribs. Alternatively, the first connector 510 can include any number of ribs.

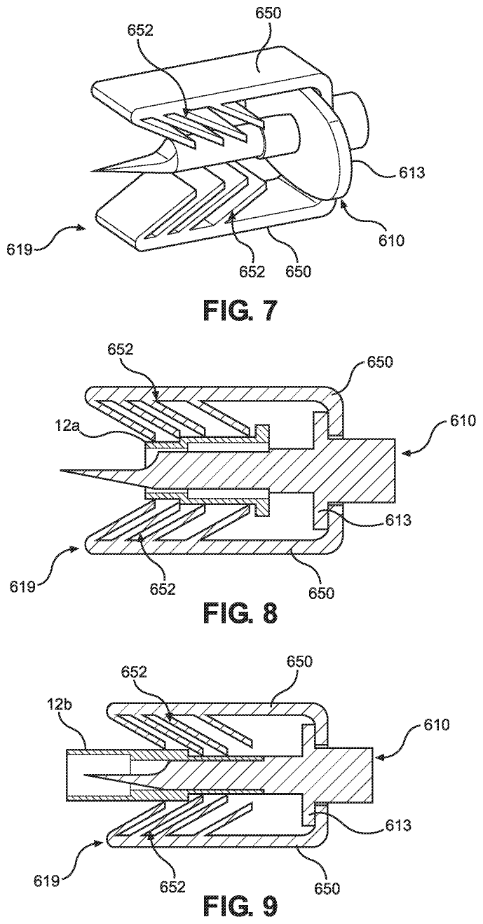

FIGS. 7-9 show views of the first connector 610 with a restrainer 619. Except for mutually exclusive features, the first connector 610 can include any or all of the features described previously in reference to the first connectors 210, 310, 410, 510 where like parts correspond to like parts, and vice versa. The restrainer 619 can be provided as an alternative to or in addition to (i.e., as a second restrainer) any of the restrainer embodiments previously described. That is, the restrainer 619 can be used with embodiments of any of the first connectors 110, 210, 310, 410, 510 as an alternative or an additional restrainer.

The restrainer 619 can be removably connected to the first connector 610. For example, the restrainer 619 can be slide onto the first connector 610 proximally from a flange 613 of the first connector 610. The restrainer 619 can include one or more arms 650, which can extend along the longitudinal direction of the first connector 610. In embodiments such as shown in FIGS. 7-9, the restrainer 619 can include two arms 650, though other combinations of arms 650 are possible. Each arm 650 can include one or more teeth 652. In embodiments such as shown in FIGS. 7-9, each arm 650 can include four teeth 652, though other combinations of teeth 652 are possible. The teeth 652 can extend angularly from the respective arm 650 towards a proximal end of the restrainer 619. According to this configuration, the teeth 652 can be slid along the port 12 of the IV bag 10 when the first connector 610 is inserted into the port 12 without interfering with the insertion. Further, the teeth 652 can grip exterior surfaces of the port 12 after the first connector 610 is inserted into the port 12 to resist disconnection of the port 12 and the first connector 610. As shown in FIG. 8, the teeth 652 can grip a rigid port 12a including at a proximal flange of the rigid port 12a. As shown in FIG. 9, the teeth 652 can grip a flexible port 12b.

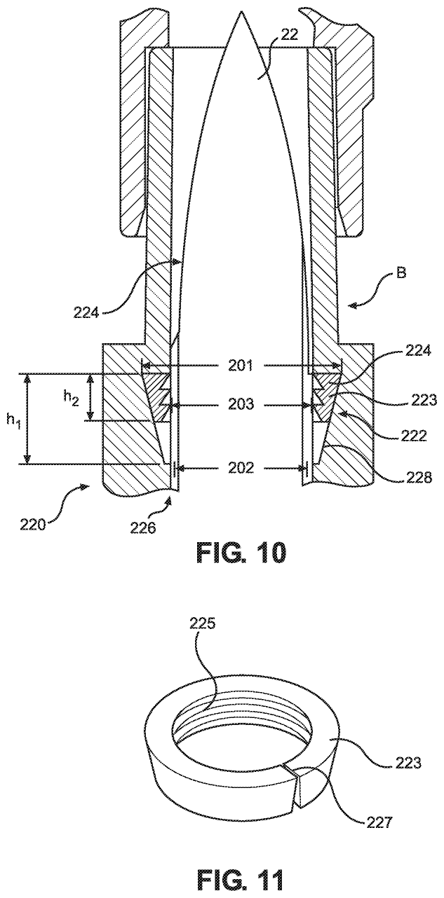

FIGS. 10-18 show embodiments of second connectors 220, 320, 420, 520, 620. Except for mutually exclusive features, the embodiments of the second connectors 220, 320, 420, 520, 620 can include any of the features previously described in reference to the second connector 120, where like parts correspond to like parts, and vice versa. Further, any of the second connectors 220, 320, 420, 520, and 620 can be used in the system 1 and can be provided as a part of the fitting 100 in the same manner described previously in reference to the second connector 120.

FIGS. 10 and 11 show aspects of the second connector 220. The second connector 220 together with the IV set spike 22 of the infusion set 20 can form the connection B. The second connector 220 can include a port 224 with an opening 226 for receiving the IV set spike 22. The port 224 can include a recess 228 that can hold an opposer 222. The recess 228 can be conical and can taper downwardly towards the opening 226. For example, as shown in FIG. 10 the recess 228 can have a first inner diameter 201 at an innermost portion of the recess 228 relative to the opening 226 and a second inner diameter 202 at an outermost portion of the recess 228 relative to the opening 226. The first inner diameter 201 can be greater than the second inner diameter 202 and the recess 228 can taper along a first height h₁ of recess 228 from the first inner diameter 201 to the second inner diameter 202. The recess 228 can taper continuously or discontinuously.

As shown in FIG. 10, the second connector 220 can include the opposer 222 held within the recess 228. The opposer 222 can include a locking ring 223 held within the recess 228. FIG. 11 shows a perspective view of the locking ring 223 shown in FIG. 10. The locking ring 223 can include inner circumferential ridges 225. The inner circumferential ridges 225 can have a third inner diameter 203 that can grip the IV set spike 22 to resist removal of the IV set spike 22 from the port 224. The locking ring 223 can have a second height h₂. The locking ring 223 can include a longitudinal slit 227. The locking ring 223 can be made of any suitable material including for example plastic and/or metal. The locking ring 223 can include a taper that is complementary to the taper of the recess 228 such that the locking ring 223 can fit securely within the recess 228. For example, the locking ring 223 can be conically shaped.

In embodiments, the second heigh h₂ of the locking ring 223 can be less than the first height h₁ of the recess 228 and accordingly the recess 228 can accommodate a translation of the locking ring 223 within the recess 228. The locking ring 223 can initially be disposed at the narrowest region of the recess 228, i.e., adjacent to the second inner diameter 202. The third inner diameter 203 of the locking ring 223 can be variable. Upon insertion of the IV set spike 22 within the port 224, the locking ring 223 can translate within the recess 228 further into the interior of the port 224 and can abut against the widest region of recess 228, i.e., adjacent to the first inner diameter 201. As the locking ring 223 translates within the recess 228, the longitudinal slit 227 can allow the locking ring 223 to expand within the recess 228, i.e., to increase the third inner diameter 203 to accommodate insertion of the IV set spike 22. Upon application of a removal force attempting to break apart connection B (e.g., second force F2 discussed above), the third inner diameter 203 can decrease such that third inner diameter 203 is less than an external diameter of IV set spike 22. For example, the third inner diameter 203 can decrease to at least 4.5 mm (+/−10%) at narrowest region of recess 228. Since the third inner diameter 203 can decrease such that the third inner diameter 203 is less than the external diameter of IV set spike 22 (i.e., 5 mm), the locking ring 223 can grip the IV set spike 22 via the inner circumferential ridges 225.

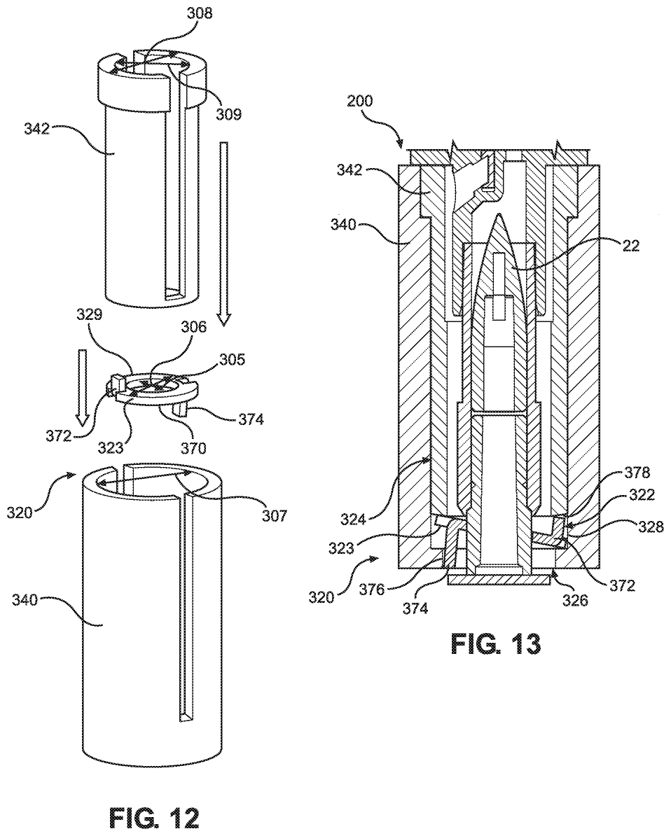

FIGS. 12 and 13 respectively show exploded and cross-section view of the second connector 320. Except for mutually exclusive features, the second connector 320 can include any or all of the features described previously in reference to the second connector 220 where like parts correspond to like parts, and vice versa. For example, the second connector 320 can include a port 324 with an opening 326 for receiving IV set spike 22. Port 324 can include a recess 328 that can hold opposer 322. Opposer 322 can include a locking ring 323 held within recess 328. Locking ring 323 can grip the IV set spike 22 to resist removal of IV set spike 22 from port 324.

The locking ring 323 can be disc-shaped and can have an inner diameter 305 and an external diameter 306. Inner diameter 305 can be sized to accommodate insertion of IV set spike 22. The external diameter 306 can be sized to fit into the opening 326. The locking ring 323 can have a topside 329 and a bottom side 370. In embodiments, the topside 329 has a top projection 372 that is substantially perpendicular to a plane defined by the topside 329. The bottom side 370 can include a bottom projection 374 that is substantially perpendicular to the plane of the bottom side 370 and in a 180° direction opposite the top projection 372 and on opposite portions of the locking ring 323. The bottom projection 374 and the top projection 372 can have different lengths. For example, in the embodiments shown in FIGS. 12 and 13 the bottom projection 374 can be longer than the top projection 372.

The locking ring 323 can be positioned inside a first holder 340 having an inner diameter 307 that is sized to accommodate the external diameter 306 of the locking ring 323. A second holder 342 can have external diameter 308 that is sized to fit into the inner diameter 307 of first holder 340. The second holder 342 can be positioned on top of (i.e., distally with respect to) the locking ring 323 within the first holder 340. In embodiments, the first holder 340 can include guard rails on an outer surface of the body.

The second holder 342 can have an inner diameter 309 that is larger than the external diameter of IV set spike 22. Upon insertion of IV set spike 22 within port 324, the locking ring 323 angles such that the bottom projection 374 is in contact with an interior bottom surface 376 of the first holder 340 and the top projection 372 is in contact with a bottom edge 378 of the second holder 342. As the bottom projection 374 can be longer than the top projection 372, the locking ring 323 angles such that the locking ring 323 retains the inserted IV set spike 22 at an inner edge of the locking ring 323 adjacent to the bottom projection 374 and at an inner edge of the locking ring 323 adjacent to the top projection 372 and wedges the IV set spike 22 into a position that restrains axial movement.

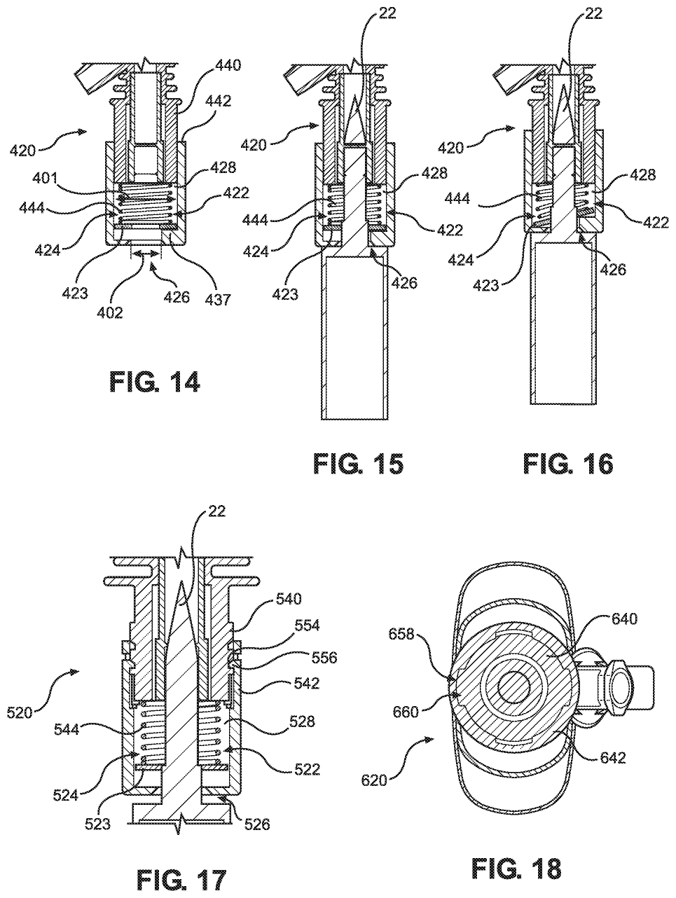

FIGS. 14-16 show cross section views of the second connector 420. Except for mutually exclusive features, the second connector 420 can include any or all of the features described previously in reference to the second connectors 220, 320 where like parts correspond to like parts, and vice versa. Second connector 420 can include a port 424 defined within a first holder 440 and a second holder 442. The port 424 can include an opening 426 for receiving the IV set spike 22. The port 424 can include a recess 428 that can hold an opposer 422. The opposer 422 can include a locking ring 423 and a spring 444 held within the recess 428. The spring 444 can exert a force on the locking ring 423 and the locking ring 423 can grip the IV set spike 22 to resist removal of the IV set spike 22 from the port 424.

In embodiments, the locking ring 423 can be planar and can be positioned within the recess 428 of the second connector 420. An internal diameter 402 of the opening 426 can be smaller than an internal diameter 401 of the recess 428. The second connector 420 can have an interior step 437 projecting into the recess 428. In embodiments, the interior step 437 does not extend more than 25% of the circumference of an inner wall of second connector 420. In embodiments, the locking ring 423 has an outer circumference that is no more than 10% smaller than circumference of the inner wall of the second connector 420. The locking ring 423 can have an inner circumference opening that can accommodate the IV set spike 22. The spring 444 can positioned on top of the locking ring 423 within recess 428.

Upon insertion of the IV set spike 22 into the second connector 420 through the locking ring 423, the axial movement back out of the second connector 420 can be locked by the locking ring 423. This can occur as a result of the locking ring 423 being pushed at an angle by the spring 444 against the interior step 437 and the interior bottom of the holder. This can retain the IV set spike 22 with the now angled the inner circumference of the opening of the locking ring 423 pressing against the IV set spike 22 and wedging against the IV set spike 22 to restrain axial movement.

FIG. 17 shows a cross section view of the second connector 520. Except for mutually exclusive features, the second connector 520 can include any or all of the features described previously in reference to the second connectors 220, 320, 420 where like parts correspond to like parts, and vice versa. The second connector 520 can include a port 524 defined within a first holder 540 and a second holder 542. The port 524 can include an opening 526 for receiving the IV set spike 22. The port 524 can include a recess 528 that can hold an opposer 522. The opposer 522 can include a locking ring 523 and a spring 544 held within the recess 528. The spring 544 can exert a force on the locking ring 523 and the locking ring 523 can grip the IV set spike 22 to resist removal of the IV set spike 22 from the port 524. The first holder 540 can include a first connector 554 and the second holder 542 can include a second connector 556. The first connector 554 and the second connector 556 can be complimentary to each other and can together connect the first holder 540 and the second holder 542 together. In embodiments, the first connector 554 and the second connector 556 can be snap fittings, threads, or other known connectors.

FIG. 18 shows a cross section view of the second connector 620. Except for mutually exclusive features, the second connector 520 can include any or all of the features described previously in reference to the second connectors 220, 320, 420, 520 where like parts correspond to like parts, and vice versa. The second connector 620 can include a first holder 640 and a second holder 642. The first holder 640 can include a first stopper 658 and the second holder 642 can include a second stopper 660. The first stopper 658 and the second stopper 660 can be complimentary to each other and can engage together to prevent the first holder 640 and the second holder 642 from rotating relative to each other about a longitudinal axis of the second connector 620. The first stopper 658 and the second stopper 660 can configured and arranged such that the first holder 640 can be slide into the second holder 642 along the longitudinal axis of the second connector 620 without interference from the first stopper 658 and the second stopper 660. In embodiments, first stopper 658 and the second stopper 660 can be grooves, projections, combinations thereof, etc.

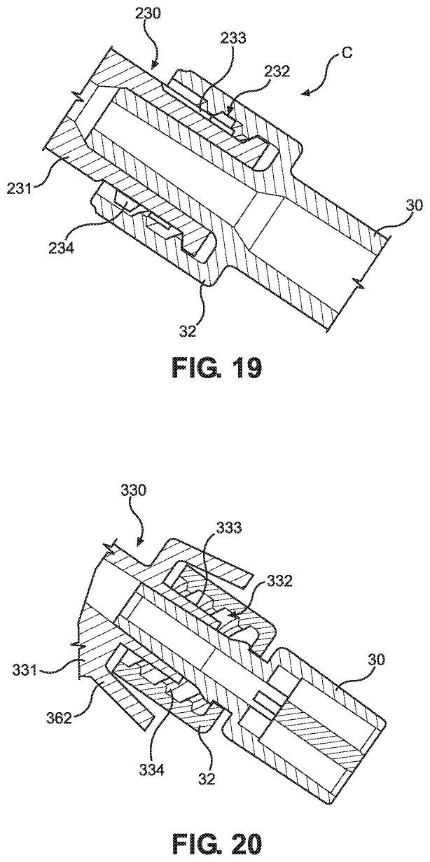

FIGS. 19 and 20 show embodiments of third connectors 230, 330. Except for mutually exclusive features, the embodiments of the third connectors 230, 330 can include any of the features previously described in reference to the third connector 130, where like parts correspond to like parts, and vice versa. Further, any of the third connectors 230, 330 can be used in the system 1 and can be provided as a part of the fitting 100 in the same manner described previously in reference to the third connector 130.

FIG. 19 shows a cross section view of the third connector 230 that together with male Luer 32 can form connection C. The third connector 230 can include a female Luer 231. The female Luer 231 can include threads that can be complementary to threads included of the male Luer 32. Accordingly, the male Luer 32 can be threaded onto the female Luer 231. The opposer 232 can be a ring 233. The ring 233 can include protrusions 234, such as fins or a ratchet having at least one angled tooth. The protrusions 234 can be arranged in a circumferential thread. The protrusions 234 can be flexible in one direction (e.g., a closing direction) and rigid in another direction (e.g., an opening direction). According to this configuration, the protrusions 234 can flex to allow a locking nut of the male Luer 32 to be threaded onto the female Luer 231. The protrusions 234 can be molded or crimped on. Once threaded on, the protrusions 234 can resist removal of the locking nut of the male Luer 32 due to rigidity of the protrusions 234 in the opening direction of the locking nut of the male Luer 32.

FIG. 20 shows a cross section view of the third connector 330. Except for mutually exclusive features, the third connector 330 can include any or all of the features described previously in reference to the third connector 230 where like parts correspond to like parts, and vice versa. For example, the third connector 330 can include a female Luer 331. The male Luer 32 can be threaded onto the female Luer 331. The third connector 330 can include an opposer 332, which can be a ring 333. The ring 333 can include protrusions 334, such as fins or a ratchet having at least one angled tooth, as discussed previously. The third connector 330 can include a guard 362. The guard 362 can surround some or all of the opposer 332. The guard 362 can protect fingers from being pinched or from interfering with the connection of the third connector 330 and the male Luer 32.

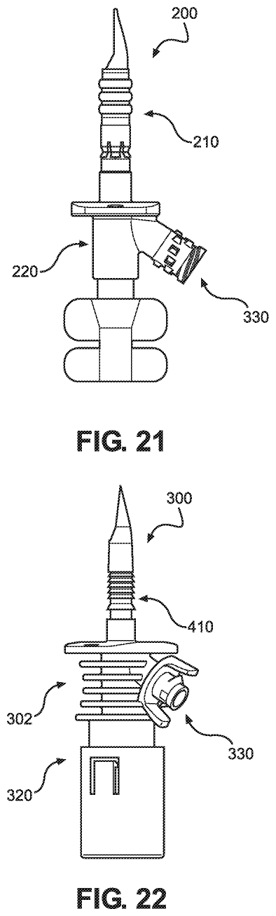

FIGS. 21 and 22 show embodiments of fittings 200, 300. Except for mutually exclusive features, the embodiments of the fittings 200, 300 can include any of the features previously described in reference to the fitting 100, where like parts correspond to like parts, and vice versa. Further, any of the fittings 200, 300 can be used in the system 1. FIG. 21 shows a view of the fitting 200. The fitting 200 can include the first connector 210, the second connector 220, and the third connector 230.

FIG. 22 shows views of the fitting 300. The fitting 300 can include the first connector 410, the second connector 420, and the third connector 330. The fitting 300 can include a body 302 that defines a grip to help a user grasp the fitting 300. In embodiments, any of the previously described fittings 100, 200 can include a similar body defining a grip.

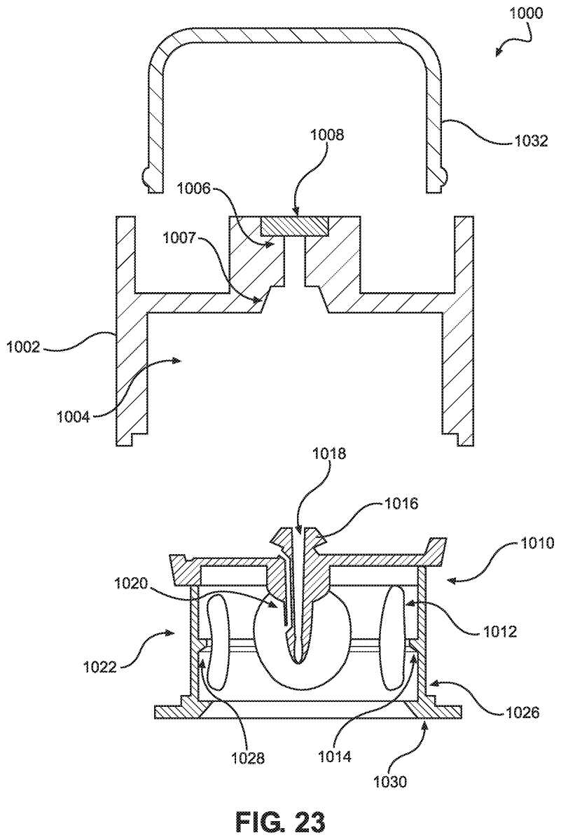

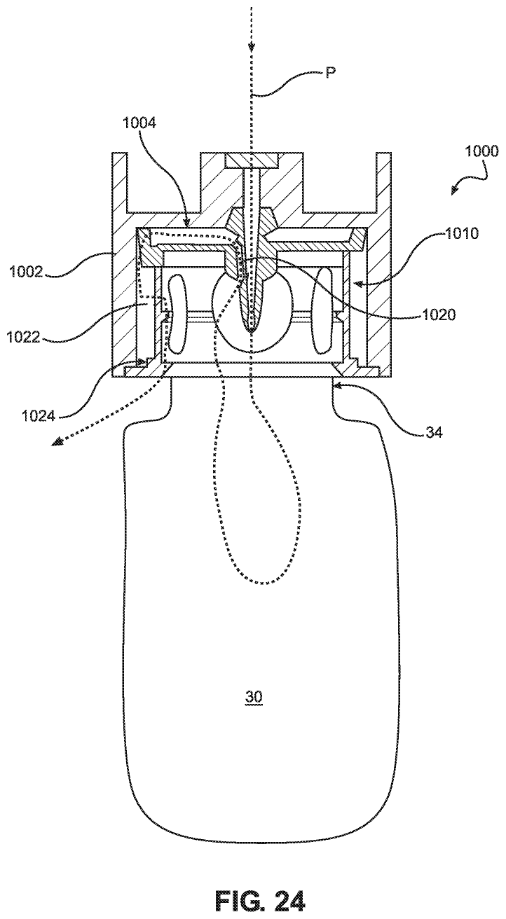

FIGS. 23 and 24 show a device 1000 for closed sterile transfer (CST). The device 1000 can include an outer body 1002. In embodiments, the outer body 1002 of the device 1000 can include a connector (e.g., a Luer lock connector) that can connect with any of the third connectors 130, 230, 330 to form the connection C and can provide CST of medicament from the medicament supply 30. Additionally or alternatively, the device 1000 can provide CST of medicament from the medicament supply 30 to any number of other devices including a syringe among other possibilities. The outer body can define an interior 1004. The outer body 1002 can include a channel 1006 that can extend through the outer body and can open at an opening 1007 into the interior 1004. The device 1000 can include a septum 1008. The septum 1008 can seal the channel 1006 of the outer body 1002. In embodiments, the septum 1008 can be split or punctured by a syringe during CST of the medicament from the medicament supply 30.

The device 1000 can include an inner body 1010 that can be contained within the interior 1004 of the outer body 1002. The inner body 1010 can define an interior 1012. The inner body 1010 can include a seal 1014 within the interior 1012 of the inner body 1010. The seal 1014 can fluidly seal an opening 34 of the medicament supply 30 (e.g., a container, a vial, etc.). The inner body 1010 can include a projection 1016. The projection 1016 can extend through the seal 1014. The projection 1016 can include a first channel 1018 that fluidly connects the channel 1006 of the outer body 1002 and the interior 1012 of the inner body 1010. The projection 1016 can include a second channel 1020 that can fluidly connect the interior 1004 of the outer body 1002 and the interior 1012 of the inner body 1010. In embodiments, the first channel 1018 can be used to extract the medicament from the medicament supply 30 and the second channel 1020 can be a part of a pathway P that can serve as a vent for gas (e.g., air) during extraction of the medicament.

The inner body 1010 can also include a filter 1022. The filter 1022 can extend circumferentially around a central axis of the inner body 1010 and can be arranged within the interior 1004 of the outer body 1002 when the inner body 1010 is assembled together with the outer body 1002. According to this configuration, the filter 1022 can filter gas or other substances exiting from the interior of the medicament supply 30 (e.g., via the pathway P) and to provide CST. Because the filter 1022 can be arranged circumferentially around the central axis of the inner body 1010, the device 1000 can have a compact form factor, which can for example improve storage and usability of the device 1000 and can improve disposability since the device 1000 takes up less space than if the filter was provided externally.

In embodiments, the filter 1022 can include hydrophobic media, which can reduce or prevent fluid from passing through the filter 1022. In embodiments, the filter 1022 can be charged (e.g., with a charge-based coating), which can attract and filter complementarily charged particles. In embodiments, the filter 1022 can include pores. The pores can for example have a size of 0.2 μm or less. In embodiments, the filter 1022 can include a material that inhibits biological attachment and/or proliferation including for example PTFE, among other possibilities. In embodiments, the filter 1022 can be pleated, which can increase the surface area of the filter 1022 and improve filtration. In embodiments, the filter 1022 can be resilient and can exert a force on the seal 1014 to fluidly seal the opening 34 of the medicament supply 30.

As shown in FIG. 24, the inner body 1010 and the outer body 1002 can together define a pathway P for gas to exit from the interior 1012 of the inner body 1010. The pathway P can extend through the second channel 1020 of the inner body 1010, through the interior 1004 of the outer body 1002, through the filter 1022, and out of an outlet 1024 of the device. According to this configuration, the filter 1022 can reduce or prevent leakage of medicament and/or vapors from the medicament supply 30 and can provide CST of medicament/other substances from the medicament supply 30. Since the filter 1022 is arranged internally (e.g., circumferentially around the inner body 1010) gas can be routed internally and the overall form factor of the device 1000 can be improved.

In embodiments, the medicament supply 30 can include a plastic material that forms the opening 34. The plastic material can include for example cyclic olefin polymer and/or cyclic olefin copolymer. The device 1000 can connect to the medicament supply 30, including embodiments of the medicament supply 30 that include the plastic material forming the opening 34, in a manner that allows for CST of the medicament within the medicament supply 30. The device 1000 can also comprises plastics such as for example cyclic olefin polymer and/or cyclic olefin copolymer, though other additional or alternative materials are possible.

For example, the device 1000 can include a connector 1026 that can connect with the opening 34 of the medicament supply 30 and that can enable CST of a substance from the medicament supply 30. In embodiments, the connector 1026 can be pressed onto the opening 34 of medicament supply 30. In embodiments, the connector 1026 can be screwed onto the opening of the medicament supply 30. The connector 1026 of the device 1000 can include, for example, a first lock 1028 and a second lock 1030. The first lock 1028 and a second lock 1030 can be complementary to a connector (e.g., a flange, nubs, tabs, ridges, etc.) of the opening 34 to connect to the opening 34. In embodiments, the seal 1014 can be resilient and can form a component of the connector 1026. In embodiments, the filter 1022 can be resilient and can form a component of the connector 1026. Additionally or alternatively, the filter 1022 can include a separate structure (e.g., a ring) that can be overmolded to exert a force on the opening 34 to seal the opening 34.

In embodiments, the device 1000 can include a cap 1032. The cap 1032 can be removably connected to the outer body 1002 to cover the septum 1008.

It will be appreciated that the foregoing description provides examples of the invention. However, it is contemplated that other implementations of the invention may differ in detail from the foregoing examples. All references to the invention or examples thereof are intended to reference the particular example being discussed at that point and are not intended to imply any limitation as to the scope of the invention more generally. All language of distinction and disparagement with respect to certain features is intended to indicate a lack of preference for those features, but not to exclude such from the scope of the invention entirely unless otherwise indicated.

1. A closed-system liquid transfer device configured for use with each of an infusion liquid container containing an infusion liquid and having a port for administering the infusion liquid, a medicament supply containing a medicament and having a male Luer, and an infusion set including an IV set spike, the closed-system liquid transfer device comprising:
a body;
an IV set spike at a first end of the body configured to be fluidly connected to the port of the infusion liquid container;
a port at a second end of the body comprising a locking ring to sealingly receive the IV set spike of the infusion set and to resist a removal force applied to the port of the closed-system liquid transfer device or to the IV set spike of the infusion set; and
a female Luer at a third end of the body configured to be fluidly connected to the male Luer of the medicament supply.

2. The closed-system liquid transfer device of claim 1, wherein the locking ring has a plurality of circumferential ridges on an inner surface of the locking ring, and the locking ring is provided within a recess of the port of the closed-system liquid transfer device.

3. (canceled)

4. The closed-system liquid transfer device of claim 2, wherein the recess is tapered, and translation of the locking ring towards a narrow portion of the recess reduces a diameter of the locking ring.

5-6. (canceled)

7. The closed-system liquid transfer device of claim 1, wherein the female Luer comprises a plurality of protrusions that are configured to resist rotation of the male Luer in an opening direction.

8. (canceled)

9. The closed-system liquid transfer device of claim 1, wherein the IV set spike of the closed-system liquid transfer device comprises a restrainer having an outwardly projecting trailing edge defining a plurality of barbs.

10. (canceled)

11. The closed-system liquid transfer device of claim 9, wherein the IV set spike of the closed-system liquid transfer device comprises:
a tip; and
one or more ribs between the tip and the restrainer.

12. The closed-system liquid transfer device of claim 9, wherein the IV set spike of the closed-system liquid transfer device includes an abutment, and an opposing end of the restrainer relative to the outwardly projecting trailing edge is adjacent to the abutment.

13. The closed-system liquid transfer device of claim 1, further comprising:
a first lumen fluidly connecting an opening of the female Luer with a first distally disposed opening of the IV set spike of the closed-system liquid transfer device, the first lumen being configured to provide fluid communication from the medicament supply via the female Luer to the infusion liquid container that is separated from the port of the closed-system liquid transfer device; and
a second lumen fluidly connecting the port of the closed-system liquid transfer device with a second distally disposed opening of the IV set spike of the closed-system liquid transfer device, the second lumen being configured to provide fluid communication from the infusion liquid container to the port of the closed-system liquid transfer device including liquid received from the medicament supply via the first lumen.

14. The closed-system liquid transfer device of claim 1, wherein the locking ring has a topside and a bottom side, wherein the topside has a top projection that is substantially perpendicular to a plane of topside and the bottom side has a bottom projection that is substantially perpendicular to a plane of the bottom side and in a 180° direction opposite the top projection and on opposite portions of the locking ring.

15. (canceled)

16. The closed-system liquid transfer device of claim 1, wherein the locking ring is planar and positioned within a holder having an opening at its bottom sized to accommodate insertion of the IV set spike of the infusion set, wherein the holder has an inner wall and an interior step, wherein the inner wall a larger diameter than a diameter of the opening and the interior step is between the opening and the inner wall, the locking ring is on top of the interior step, and a spring is on top of the locking ring.

17. (canceled)

18. The closed-system liquid transfer device of claim 16, wherein:
the holder is a first holder,
the closed-system liquid transfer device further comprises a second holder received within the first holder, and
the first holder comprises a first connector and the second holder comprises a second connector that is complementary to the first connector and that is configured to connect with the first connector to connect the first holder and the second holder.

19. (canceled)

20. The closed-system liquid transfer device of claim 18, wherein the first holder comprises a first stopper and the second holder comprises a second stopper that is complementary to the first stopper and that is configured to engage with the first stopper to prevent relative rotation between the first holder and the second holder.

21. (canceled)

22. A kit comprising:
the closed-system liquid transfer device of claim 1;
the infusion liquid container;
the medicament supply; and
the infusion set.

23. (canceled)

24. A closed-system liquid transfer device configured for use with each of an infusion liquid container containing an infusion liquid and having a port for administering the infusion liquid, a medicament supply containing a medicament and having a male Luer, and an infusion set including an IV set spike, the closed-system liquid transfer device comprising:
a body;
an IV set spike at a first end of the body configured to be fluidly connected to the port of the infusion liquid container;
a port at a second end configured to sealingly receive the IV set spike of the infusion set; and
a female Luer at a third end of the body configured to be fluidly connected to the male Luer of the medicament supply, the female Luer comprising a plurality of a plurality of protrusions that are configured to resist rotation of the male Luer in an opening direction.

25. The closed-system liquid transfer device of claim 24, wherein the plurality of protrusions are arranged in a circumferential thread.

26. (canceled)

27. The closed-system liquid transfer device of claim 24, wherein the IV set spike of the closed-system liquid transfer device comprises a restrainer having an outwardly projecting trailing edge defining a plurality of barbs.

28. (canceled)

29. The closed-system liquid transfer device of claim 27, wherein the IV set spike of the closed-system liquid transfer device comprises:
a tip; and
one or more ribs between the tip and the restrainer.

30. The closed-system liquid transfer device of claim 27, wherein the IV set spike of the closed-system liquid transfer device includes an abutment, and an opposing end of the restrainer relative to the outwardly projecting trailing edge is adjacent to the abutment.

31. The closed-system liquid transfer device of claim 27, further comprising:
a first lumen fluidly connecting an opening of the female Luer with a first distally disposed opening of the IV set spike of the closed-system liquid transfer device, the first lumen being configured to provide fluid communication from the medicament supply via the female Luer to the infusion liquid container that is separated from the port of the closed-system liquid transfer device; and
a second lumen fluidly connecting the port of the closed-system liquid transfer device with a second distally disposed opening of the IV set spike of the closed-system liquid transfer device, the second lumen being configured to provide fluid communication from the infusion liquid container to the port of the closed-system liquid transfer device including liquid received from the medicament supply via the first lumen.

32. A kit comprising:
the closed-system liquid transfer device of claim 24;
the infusion liquid container;
the medicament supply; and
the infusion set.

33-47. (canceled)
